(19) United States
(12) Patent Application Publication
(10) Pub. No.: US 2025/0316184 A1
(43) Pub. Date: Oct. 9, 2025

(54) TRAINING PLATFORM

(71) Applicant: VIRTUALISURG, Paris (FR)

(72) Inventors: Tom LORENT BOURDO, Paris (FR); Nicolas MIGNAN, Paris (FR)

(73) Assignee: VIRTUALISURG, Paris (FR)

(21) Appl. No.: 18/865,453

(22) PCT Filed: May 15, 2023
(86) PCT No.: PCT/FR2023/050695

§ 371 Date: Nov. 13, 2024

(30) Foreign Application Priority Data
May 13, 2022 (FR) FR2204583

(51) Int. Cl. G09B 19/00; A61B 34/00; A61B 34/10; A61B 34/30; A61B 90/00; G06F 3/01

(52) U.S. Cl. CPC G09B 19/00; A61B 34/10; A61B 34/30; A61B 34/76; A61B 90/36; G06F 3/011; G06F 3/016; A61B 2034/104; A61B 2090/365; A61B 2560/0223

(57) ABSTRACT

A surgical training platform including a display unit for displaying the virtual environment, a calibration module connected to the display unit, a training module with a haptic controller, a control system configured to identify the different modules, analyse the data related to the movement(s) of each tool connected to the haptic controller, generate the virtual environment, interface each virtual surgical element with a corresponding real element. All of modules are configured to be reversibly attached to each other in a known configuration, each haptic controller includes a connection system configured to reversibly mechanically connect a surgical training tool, each haptic controller being further configured to measure each movement in space of the surgical training tool once the latter is connected to the haptic controller. The control system further includes a system for recognizing the tool.

### FIELD OF THE INVENTION

The present invention relates to a modular surgical training platform. Hence, the present invention relates to the field of education and teaching tools, methods and equipment. More particularly, the invention relates to a device for training for a surgical procedure, intended for training surgeons.

### PRIOR ART

To date, most surgical trainings are carried out under real conditions, on patients, by means of a surgical companion. This method requires considerable human resources, involves significant hardware constraints and might generate a considerable stress for the student which might lead to difficulties in concentration and/or memorisation.

Alternatives exist, like, for example, Pelvitrainer EoSim SurgTrac® or some sessions on animals. However, these trainings/methods are accessible only to a small number of surgery interns and have some obvious limitations: the Pelvitrainer is a simple box in which trocars and a camera are inserted with the possibility of practicing sutures on inert materials like foam. The animal model has obvious problems in terms of training quality because the anatomical similarities/correlations with humans are limited. The animal model also poses ethical problems.

Hence, the present invention aims to provide a safe, practical, accurate, realistic, easy-to-use and easily available training device, allowing give any surgery student a chance to train in a safe environment without any risk of injuring himself/herself, injuring a patient or an animal.

### SUMMARY

Hence, this invention relates to a modular surgical training platform configured to interface a virtual environment comprising at least one virtual surgical element, the training platform comprising a virtual reality display unit configured to display, to a user, the virtual environment, a calibration module connected to the virtual reality display unit, at least one training module, each training module including a haptic controller, a control system configured to identify the different modules connected together, generate the virtual environment, interface each movable virtual surgical element of the virtual environment with a corresponding real element. The invention is characterised in that all of the modules are configured to be reversibly attached to each other in a known configuration. The present invention is also characterised in that each haptic controller includes a connection system configured to reversibly mechanically connect a surgical training tool, each haptic controller being further configured to measure each movement in space of the surgical training tool once the latter is connected to the haptic controller. The present invention is also characterised in that the control system further includes a system for recognising the surgical training tool configured to obtain identification information specific to the connected surgical training tool, and communicate the identification information to the control system, so that the control system recognises each surgical training tool connected to the haptic controller. The invention is also characterised in that the control system generates a virtual image of each surgical training tool connected to the haptic controller. The invention is also characterised in that the control system is configured to receive and analyse the data related to the movement(s) of each surgical training tool connected to the haptic controller, and reproduce each movement of the surgical training tool connected to the haptic controller into a corresponding virtual movement of its virtual image in the virtual environment. Finally, the invention is characterised in that each virtual movement is made visible to the user by the display unit.

Thus, the solution allows achieving the aforementioned objective. In particular, the platform according to the present invention being modular, the different modules composing the haptic control platform of the virtual surgery elements can be easily interfaced with one another. This modularity also allows adapting the platform to the different surgical exercises that are proposed, by adding and removing elements of the latter. It also allows adapting the platform to the preferences of the user; for example, if the user is left-handed, the calibration module may be placed to the right so as not to hinder his/her movements. The freedom of arrangement of the surgical tools also corresponds more to the reality of the surgery exercise. Indeed, under real conditions, the practitioner can place his/her tools as he/she wishes to facilitate his/her work.

The physical connection enables the identification of the different modules and/or the transmission of information on the movement of the haptic elements.

The specific identification information may be a voltage measured at the level of a voltage divider bridge specific to the connected surgical tool. Alternatively, the specific identification information may be contained in an electronic component such as an electronic chip.

The platform according to the invention may comprise one or more of the following features, considered separately or combined with one another:
the control system may include:
   a measuring unit configured to:
      identify the connected surgical training tools and/or modules,
      gather the movement data of the connected surgical training tool,
   a central unit configured to:
      generate the virtual environment,
      receive and analyse the data related to the movement(s) of each connected surgical training tool,
      interface each movable virtual surgical element of the virtual environment with a corresponding surgical training tool,
the measuring unit may be part of the training module,
each module may comprise a base having a specific shape, the shapes of the different bases of the different modules being complementary to one another, so as to obtain a stable and adapted interlocking of the different modules,
the connection system of the haptic controller could allow connecting at least two different surgical training tools,
the connection system may comprise at least one key-lock type locking connector configured to be fastened to the haptic controller and to any surgical training tool so as to ensure the removable connection thereof,
the connection system may comprise a first connector fastened to the haptic controller and configured to removably cooperate with a corresponding second connector fastened to the surgical training tool,
the haptic controller of the training module may comprise a swivel arm robot, the swivel arm robot having a free end intended to cooperate with the connection system,
the control system may be further configured to generate a feedback signal enabling the haptic controller to generate a haptic feedback when at least two virtual elements interact in the virtual environment.

Another object of the present invention is a surgical training kit comprising a modular surgical training platform according to any one of the technical features listed hereinabove and at least one surgical training tool configured to be connected to the haptic controller of the platform.

### BRIEF DESCRIPTION OF THE FIGURES

The invention will be better understood, and other aims, details, features and advantages thereof will appear more clearly upon reading the following detailed explanatory description, of embodiments of the invention given for illustration, of merely illustrative and non-limiting examples, with reference to the appended drawings:

FIG. 1 is a perspective overview of a dismounted platform according to the present invention,

FIG. 2 is a perspective overview of a dismounted platform according to the present invention, shown with two different surgical training tools each able to be connected to the platform,

FIG. 3 is a view similar to FIG. 2 at a different angle,

FIG. 4A is a perspective view of a training platform mounted according to the invention, connected to a surgical training tool, including one single training module,

FIG. 4B is a perspective view of a training platform mounted according to the invention, including two training modules, connected to two surgical training,

FIG. 5 is a perspective view of a kit according to the present invention, handled by a user according to a first embodiment,

FIG. 6 is a perspective view of a kit according to the present invention, handled by a user according to a second embodiment,

FIG. 7 is an exploded view of a training module,

FIG. 8A is a perspective view of a calibration element associated with the display unit according to the invention,

FIG. 8B is a perspective view of the calibration module,

FIG. 9A is a perspective view of a haptic controller connector according to the invention,

FIG. 9B is a perspective view of a surgical training tool connector according to the invention,

FIG. 10 is a perspective view of a surgical training tool,

FIG. 11 is a perspective view of an example of a virtual environment according to the invention,

FIG. 12 is a perspective view of an example of an anatomical module according to the invention,

FIG. 13 is a perspective view of an example of a storage module according to the invention,

FIGS. 14A and 14B are perspective views of an example of a complementary tooling module according to the invention.

### DETAILED DESCRIPTION

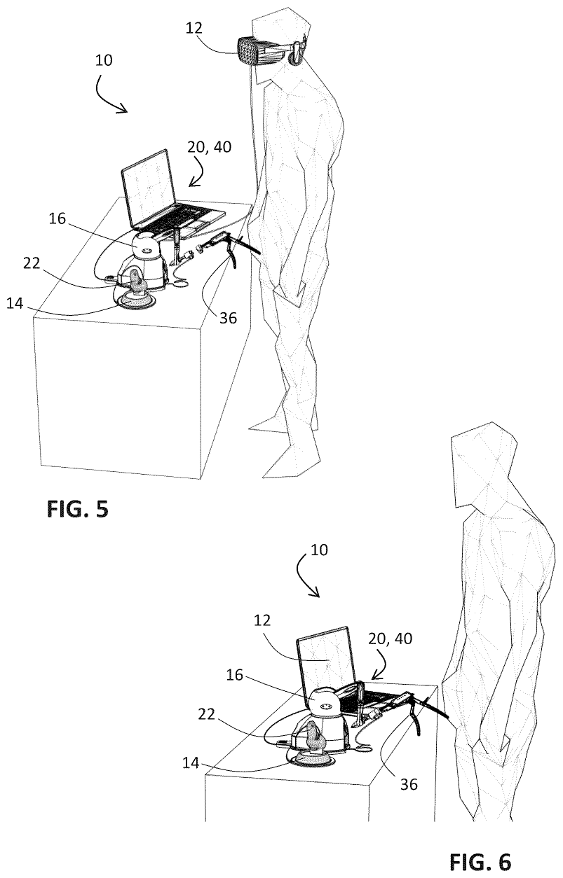

As shown in FIGS. 5 and 6, the present invention relates to a modular surgical training platform 10 configured to interface a virtual environment 100 comprising at least one movable virtual surgical element 102 (cf. FIG. 11). This virtual environment also comprises a virtual patient 104 and different decorations elements 106. Thus, a user handling the modular platform 10 interacts with the virtual environment 100 in which all kinds of surgical operations are possible.

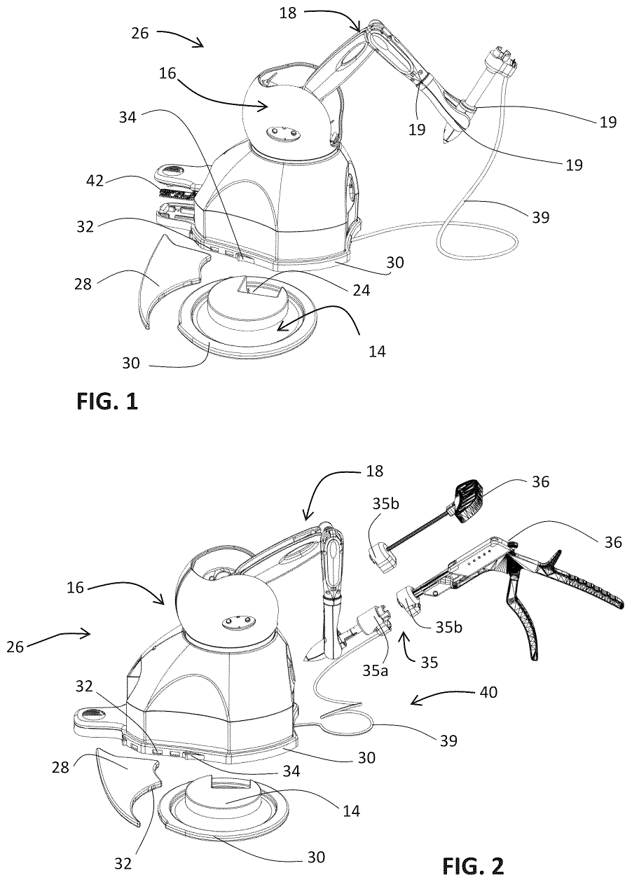

As shown in particular in FIGS. 1 and 5, the modular platform 10 according to the present invention comprises:
a virtual reality display unit 12 configured to show/display, to the user, the virtual environment 100,
a calibration module 14 connected to the virtual display unit 12, configured to align the virtual environment 100 on the physical reality of a user handling the platform 10 according to the present invention,
at least one training module 16, each training module including a haptic controller 18,
a control system 20.

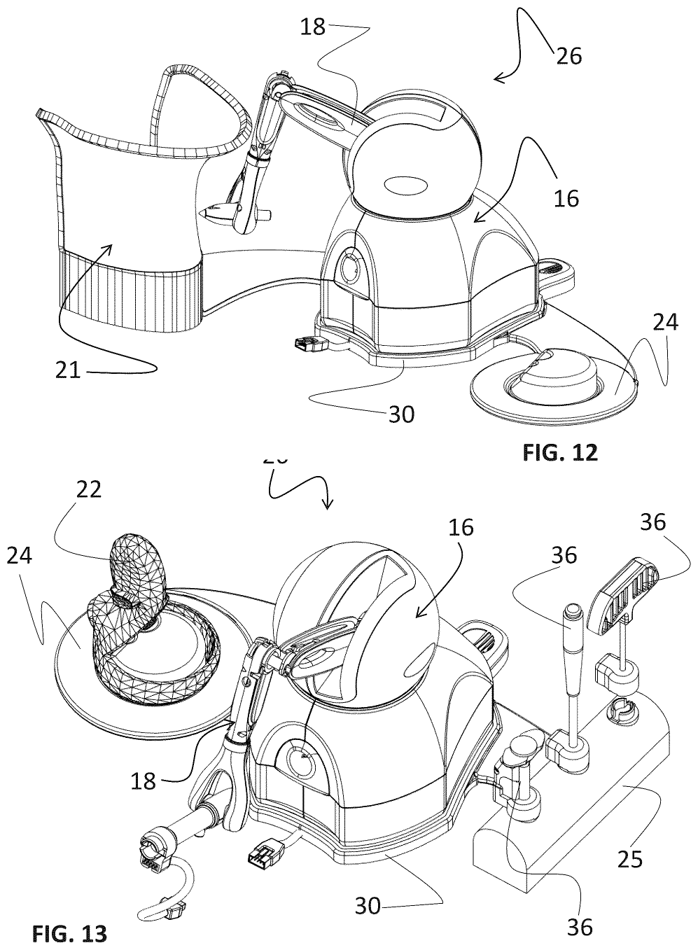

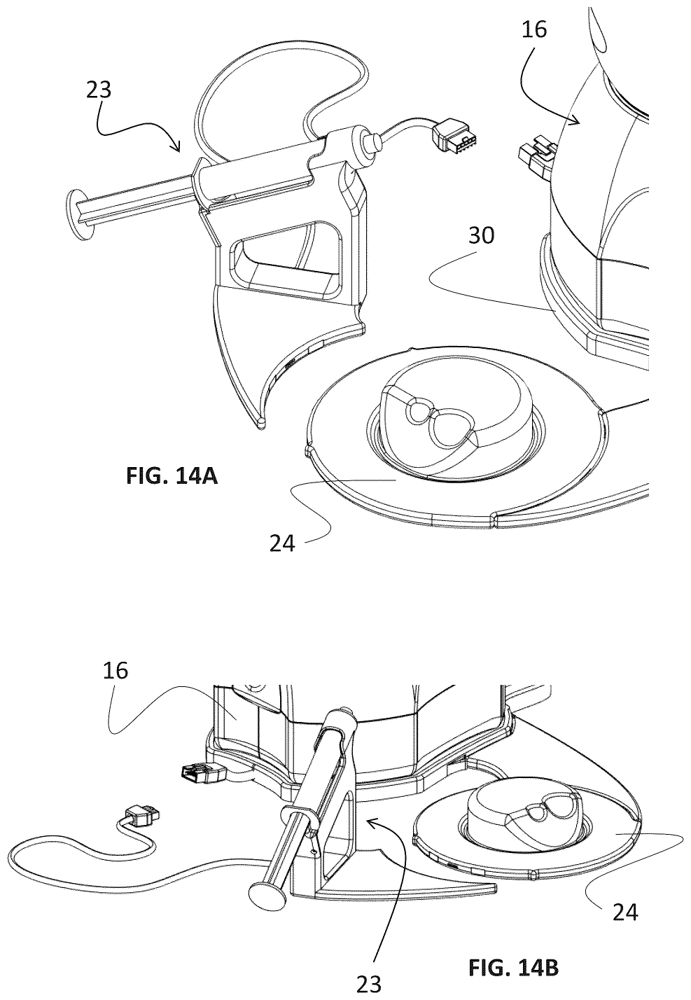

In some embodiments, the modular platform 10 may further comprise one or more optional module(s) 21, 23, 25:
at least one anatomical module 21 (cf. FIG. 12),
at least one complementary tooling module 23 (cf. FIGS. 14A and 14B),
at least one storage module 25 (cf. FIG. 13).

In the context of the present invention, the display unit 12 makes the link between the different training modules 16, the possible anatomical 21, complementary tooling 23, storage 25 modules (optional modules 21, 32, 25) and the virtual environment 100. Indeed, the training module(s) 16 and the optional modules 21, 23, 25 are the only element(s) that are handled by the user and the rendering of these handling operations is visible only in the virtual environment 100.

In general, in the present application, the module concept refers to an independent functional element forming a clearly delimited and defined object, dissociable from the other independent functional elements each of which, in turn, forms a clearly delimited and defined object. Thus, each module may be considered as a standalone entity from a functional perspective, i.e. each module ensures a specific function and is designed to ensure it directly upon connection thereof with the training module 16 directly or indirectly. Thus, each module mechanically relies on itself to perform the function for which it is designed, even though each module needs to be supplied with current in order to be usable and that the modules function only once connected together. Each module 14, 16, 21, 23, 25 is composed of different parts or elements, for example made of plastic, assembled together, so as to make up this standalone unit.

In some embodiments of the training platform 10, it is possible to find two or more training modules 16 and each of these training modules 16 can operate without the other. This is also true for the possible anatomical 21, complementary tooling 23, storage modules.

Thus, each training module 16 is a standalone entity including several parts or elements made of plastic (or made of another material) assembled together. These parts may be 3D printed. All of these parts and elements will be described throughout the present description, in connection with the different functions and technical features of the console 10 according to the present invention.

The calibration module 14 will be detailed hereinbelow in the present description.

The at least one anatomical module 21 is a standalone entity according to the definition hereinabove representing and/or rendering in 3D all or part of an anatomical portion of a patient. Each anatomical module 21 is designed so as to enable a tactile feedback and even, according to the embodiments, a haptic feedback, when the user interacts with the latter. Each anatomical module 21 may comprise one or more element(s) made of silicone, for example. Each anatomical module 21 allows giving, to the user, the impression of interacting with an external or internal anatomical portion of a patient. For example, in the case of a simulation of an aesthetic surgery at the level of the lips of a patient, it might be interesting that the user could also interact with a reproduction of the nose of said patient. This enables the user to better find his/her way on the simulation of the face of the patient being operated. In other embodiments, the at least one anatomical module 21 allows materialising an organ close to the area to be operated.

In some embodiments, the at least one anatomical module 21 may be positioned so as to alter, limit and/or hinder the movements of the user when the latter interacts with the haptic controller 18 of a training module 16. This inconvenience allows enhancing realism during the use of the console 10. The at least one anatomical module 21 may also be positioned above a training module 16 and simulate the skin of a patient.

The at least one complementary tooling module 23 may comprise all or part of a tooling likely to be present in an operating room or all or part of a surgical tool that is necessary during a specific surgical procedure but which does not intervene directly on the body of the patient of the simulation, like for example:
the regulating knob of a gas injector during a laparoscopic post-intervention verification, or
the syringe being used to inject a liquid by catheter into the body of a patient, etc. (cf. FIGS. 14A and 14B)

The at least one storage module 25 will be detailed later on.

Thus, each movable virtual surgical element 102 and each virtual movement of each of these movable virtual surgical elements 102 present in the virtual environment 100 is made visible to the user by the display unit 12.

More particularly, the display unit 12 (shown in FIGS. 5 and 6) may be an element that is fixed in space (such as a screen) or an element that is mobile in space, for example, configured to be carried by the user during the surgery operation. The display unit 12 may include several displays, enabling several users to view the virtual environment 100. The different displays may be mobile or fixed. More specifically, as shown in FIGS. 5 and 6, the display system 12 may be a screen placed on a surface proximate to or at a distance from the different modules 14, 16, 21, 23, 25. In another embodiment, the display unit 12 may be a virtual reality headset, adjustable to the user and capable of providing an audio feedback. More particularly, it may consist of an HP reverb® headset having two screens with a 2,160×2,160 pixels resolution. Each screen has a display frequency of 90 Hz. The display unit 12 is connected to the control system 20 preferably by a cable (for example a displayport or hdmi cable).

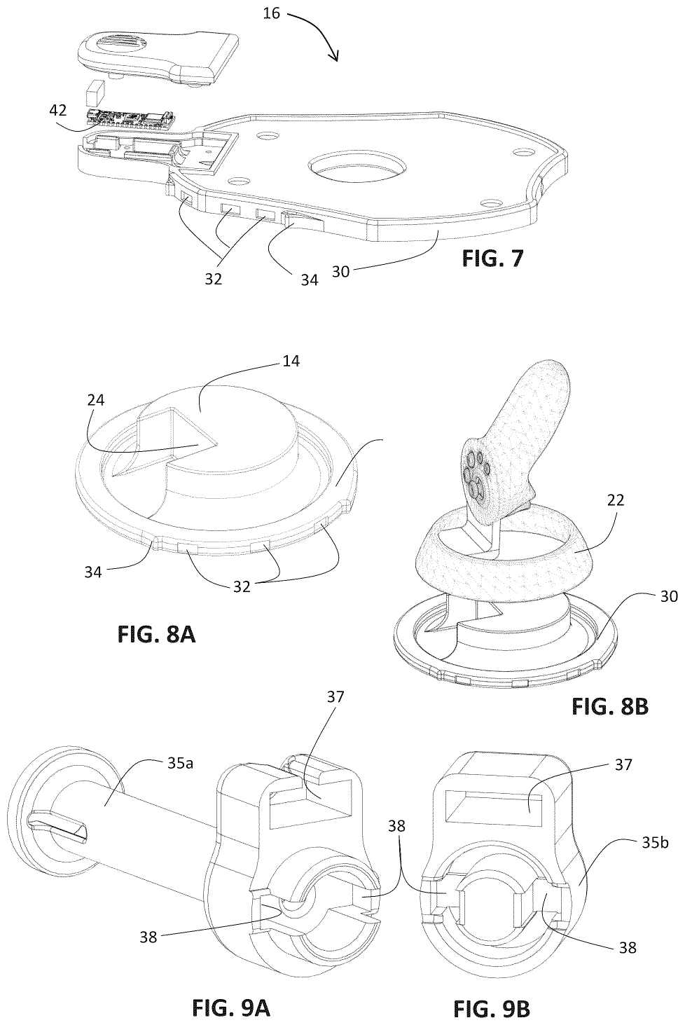

In a manner known per se, the display unit 12 is associated with a mobile calibration tool 22 (cf. FIG. 8B). The mobile calibration tool 22 may be in the form of a conventional joystick, for example, as illustrated in FIG. 8B but it could also be in a different form.

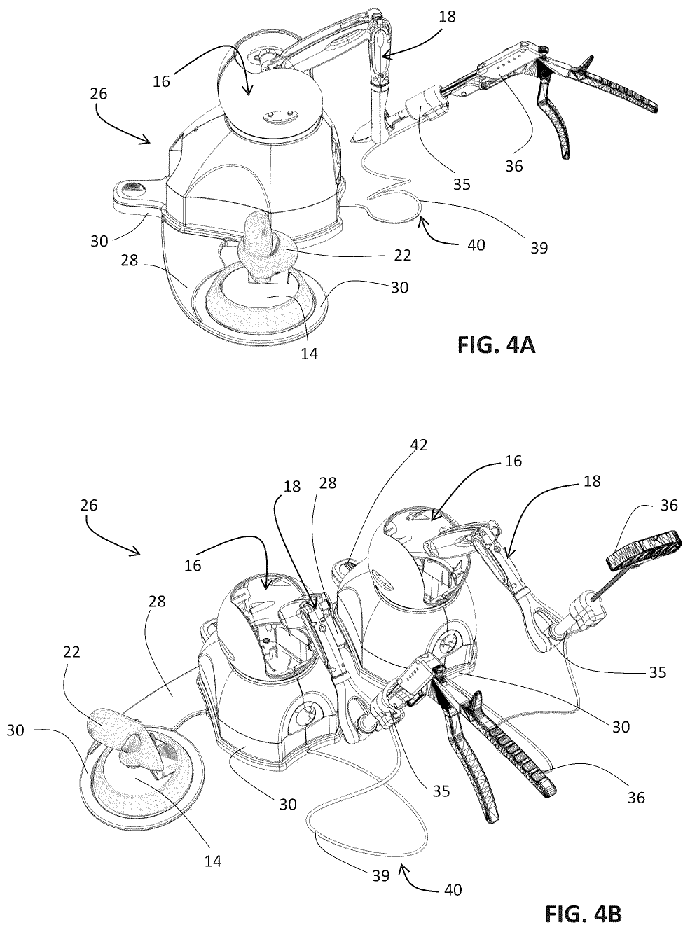

Like all of the other modules of this invention, the calibration module 14 is an independent part shown in FIG. 8A. As shown in FIG. 8B, the calibration module 14 includes an imprint 24 complementary to the mobile calibration tool 22. Thus, the calibration module 14 allows positioning the mobile calibration tool 22 associated with the display unit 12 at a known and fixed distance from the training module 16, in particular from the haptic controller 18 of the latter (cf. FIG. 4A). Preferably, the calibration module 14 is made of plastic. Preferably, it is 3D printed. In the same manner as for the training modules 16, the calibration module 14 may include magnets, as will be explained in detail hereinbelow. The calibration module 14 may also be provided with an electrical connector enabling the connection of an electronic circuit for identifying the calibration module 14 by the control system 20. For this purpose, the same device is used including a voltage divider bridge as that one used for the key-lock connector of the haptic controller 18, which will be described hereinbelow. The identification of the different modules will be detailed later on.

The haptic controller 18 allowing determining the position and the relative orientation of an object attached thereto (cf. hereinbelow), the position and the orientation of this object are then obtained with respect to the mobile calibration tool 22. In the case where the display unit 12 is a mobile device configured to be carried by the user, the calibration module 14 also allows locating the user with respect to the training module 16 and to the possible anatomical 21, complementary tooling 23, storage 25 modules. Moreover, the position of the mobile calibration tool 22 with respect to the display unit 12 being known, it is then possible to determine the position and the orientation of the object connected to the haptic controller 18 with respect to the user who carries the display unit 12 (cf. FIGS. 5 and 6).

Similarly, the different training modules 16 connected together or to the calibration module 14 and the possible optional modules 21, 23, 25 may be positioned and located by the display unit 12, since that once the different modules 14, 16, 21, 23, 25 are connected together, they are all at a fixed and known distance from the calibration module 14 and therefore from the mobile calibration tool 22 (cf. FIGS. 4A and 4B). The possible identification of the different modules 16, 21, 23, 25 through an electronic or computer system (cf. hereinbelow) could allow determining this distance in a “plug and play” fashion. Thus, the mobile calibration tool 22 associated with the display unit 12 serves as a calibration reference for each training module, therefore of each haptic controller 18 and therefore of each of the physical elements handled by the user of the platform 10.

In the present application, the “plug and play” concept describes a simple action, involving a limited number of gestures, preferably only one. Thus, a “plug and play” connection describes a connection that is done in one single gesture.

A shock (a sudden movement of the user or a handling error, for example) could lead to an inadvertent movement of the training module(s) 16 (or possible optional modules 21, 23, 25) and therefore of the calibration module 14 connected thereto, with respect to the display system 12. This could lead to a distortion of calibration between the virtual environment 100 and the position of the user. This could be avoided by using an electronic system including an accelerometer allowing, on the one hand, detecting this type of inadvertent movements and, on the other hand, adapting the digital positioning of the virtual environment 100 to the new position of the calibration module 14 with the mobile calibration tool 22.

The different training modules 16 may be connected together so as to form a control console 26 (cf. FIG. 4B). Thus, the control console 26 comprises at least one training module 16 (cf. FIG. 2). The control console 26 may also include one or more of the optional module(s) 21, 23, 25. The different modules 16, 21, 23, 25 of the control console 26 are connected together, directly or indirectly. More particularly, all of the modules 14, 16, 21, 23, 25 are configured to be reversibly attached to each other in a known configuration (cf. FIGS. 4A and 4B). This allows for a modularity of the platform 10 according to the invention.

The training module(s) 16 (and the possible optional modules 21, 23, 25) forming the control console 26 may be either connected together directly, or connected to each other by means of spacer modules 28 (cf. FIGS. 4A and 4B). Preferably, the spacer modules 28 are made of plastic and are preferably made by 3D printing, by layer deposition or by sintering. According to other embodiments, they may be manufactured by moulding or another process subsequently. The spacer modules 28 consist of connection parts allowing creating a known spacing (therefore a positioning) between the different modules 14, 16, 21, 23, 25 of the platform 10. Each spacer module 28 within the control console 26 has a specific shape, which may be unique or similar to that of another spacer module 28 of the control console 26.

To this end, each calibration 14 or training 16 or optional 21, 23, 25 or spacer 28 module comprises a base 30 having a specific shape (cf. FIG. 7). The shapes of the different bases 30 of the different modules 14, 16, 21, 23, 25, 28 are complementary to one another, so as to obtain a stable and adapted interlocking of the different modules 14, 16, 21, 23, 25, 28. The known aspect of the bases 30 of the different modules 14, 16, 21, 23, 25, 28 allows easily determining the relative position of the modules 14, 16, 21, 23, 25 28 in space. In some embodiments, a spacer module 28 allows positioning different modules 16, 21, 23, 25 of the control console 26 at different heights. More specifically, the at least one spacer module 28 is configured to arrange two modules 16, 21, 23, 25 on distinct horizontal planes. This allows creating a control console 26 extending according to the three dimensions in space. Preferably, the calibration 14, training 16 and optional 21, 23, 25 modules are connected together by means of the spacer modules 28 in order to increase the stability of the control console 26 and of the platform 10 as a whole when the latter is assembled.

According to the embodiment shown in FIGS. 1, 2, 7 and 8A, and that one shown in FIGS. 12, 13, 14B, the physical connection of the different modules 14, 16, 21, 23, 25, 28 to each other is achieved by means of a magnetic interlocking system enabling the easy interlocking of different modules 14, 16, 21, 23, 25, 28. More specifically, each base 30 of each module 14, 16, 21, 23, 25, 28 includes at least one magnet 32 intended to cooperate with a corresponding magnet 32 of a base 30 of a complementary module 14, 16, 21, 23, 25, 28, thereby forming a magnetic connection point. Preferably, the magnets 32 are grouped together in three at each magnetic connection point. In the case where the modules 16, 21, 23, 25 of the control console 26 and the calibration module 14 are connected together by spacer modules 28, the polarity of the magnets 32 is selected so that the spacer modules 28 and the other modules (calibration modules 14, training modules 16 and optional modules 21, 23, 25) attract each other. The presence of a magnetic interlocking system allows stabilising the interlocking between the different modules 14, 16, 21, 23, 25, 28 and limiting inadvertent disengagements in the event of an inadvertence of the user or unintentional shaking.

The physical connection may further include an electronic connector 34 enabling the electronic communication between the various modules 14, 16, 21, 23, 25, 28 and the control system 20 (cf. FIGS. 1, 2, 7 and 8A and 12, 13, 14B). Optionally, in the case where the calibration modules 14 and the training 16 and optional 21, 23, 25 modules are connected together by means of spacer modules 28, each spacer module 28 can also accommodate, at the level of each magnetic connection point, an electronic connector 34 intended to cooperate with an electronic connector of the base 30 of the calibration 14 and/or training 16 and/or optional 21, 23, 25 modules. Hence, the electronic communication between the different modules 14, 16, 21, 23, 25, 28 is ensured, whether the calibration 14 and training 16 and optional 21, 23, 25 modules are connected together directly or by means of a spacer module 28. The electronic communication is also ensured between the different modules 14, 16, 21, 23, 25, 28 in the case where the control console 26 extends in 3D and all its modules 16, 21, 23, 25 are not arranged on the same plane. In particular, this electronic communication enables the passage of current.

Each electronic connector 34 may be connected to a cable to connect the electronic connector of the corresponding connected module. For example, these electronic connectors 34 may be in the form of connectors with pin on retractable springs/pins. In some embodiments, each electronic connector 34 associated with a training module 16 (or an optional module 21, 23, 25) includes, for example, a voltage divider bridge generating a voltage specific to each training module 16 (and each possible optional module 21, 23, 25). This allows identifying each training module 16 and each possible optional module 21, 23, 25 by reading the voltage generated by the voltage divider bridge, in the case where this identification takes place electronically. In other embodiments, the electronic connector 34 is part of a more complex electronic circuit capable of engaging a digital communication (for example complying with the “UART” standard).

There are many technologies allowing identifying physical modules connected together using electronic means but, nonetheless, they are not used in a virtual reality context for surgery education.

To sum up, this electronic connection enables the control system 20 to:
identify the modules 14, 16, 21, 23, 25 by the control system 20, and/or
transmit the movement information of the haptic controller 18 (cf. hereinbelow).

According to the embodiments, this electronic connection may comprise a USB cable which connects the haptic controller 18 to the control system 20. This cable may be external to the training module 16.

The identification of the different modules 14, 16, 21, 23, 25, 28 by the control system 20 may be done according to two modes: by a so-called “electronic” (“hardware+software”) way or by a so-called “software” (“software+guidelines”) way. The so-called “electronic” way will be detailed hereinbelow and a few examples will be mentioned. The so-called “software” way for identification of the modules 14, 16, 21, 23, 25, 28 is based on preprogramming a software of the control system 20 and guiding the user during installation of the control console 26, for example by means of an installation manual which assigns a specific location to each module in the control console 26. This enables that the mounting of the control console 26 by the user places each module in a position consistent with the preprogramming of the software. The software comprises all the connection and arrangement information of the different modules 14, 16, 21, 23, 25, 28 therebetween, and thus allows mapping the control console 26 and correctly decrypting the collected information and sending the right information to the right location.

In both cases, the electronic connection, whether by simply enabling the connection of the different modules and the current passage or by also enabling the transfer of information, enables the control system 20 to identify the different modules 14, 16, 21, 23, 25, 28 of the control console 26.

In the case of a so-called “electronic” channel identification, the control system 20 includes a microcontroller itself electrically connected, through the connectors 34 and potentially the spacer modules 28, to the calibration 14 and the training 16 modules and to the optional modules 21, 23, 25. In a first embodiment/mode of operation, the different calibration 14 and/or training 16 and/or optional 21, 23, 25 modules embed a voltage divider system. The microcontroller then reads the voltage and is capable of identifying the module(s) 14, 16, 21, 23, 25 that respond(s) by this voltage. In an alternative embodiment/mode of operation, each of the calibration 14 and/or training 16 and/or optional 21, 23, 25 modules includes an electronic board 42 enabling a digital communication with the microcontroller of the training module 16. They identify each other and are capable of exchanging information regarding an action of the user but also a feedback of the control system 20 to the user (one could imagine for example a module that lights up in red if a handling error is made).

As mentioned hereinabove and as shown in FIGS. 1, 2 and 3, each training module 16 includes a haptic controller 18.

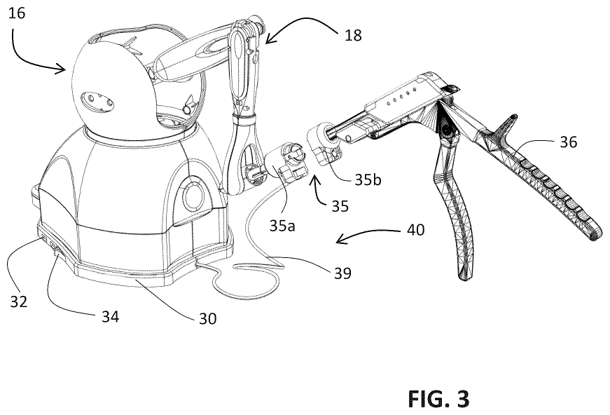

As shown in FIGS. 2 and 3. Each haptic controller 18 includes a connection system 35 configured to reversibly mechanically connect a surgical training tool 36.

To make the device for learning surgery by virtual reality proposed by the platform 10 according to the present invention more immersive and more realistic, it is interesting for the user to be able to handle physical tools to control the simulation that is displayed in the display system 12. In a manner well-known per se, the closer these physical tools are to the original surgical tool, the more immersive the simulation will be.

It is common to have to use different surgical tools during a surgical procedure. Thus, the control console 26 may comprise at least one storage module 25. Each storage module 25 has an imprint 360 of one or more surgical training tool(s) 36 in order to be able to store therein the corresponding surgical training tool(s) 36. Thus, all of the surgical training tools 36 necessary for the user to complete the surgical simulation are stored nearby. Each imprint 360 may be provided with a connection system intended to interact with the corresponding surgical training tool 36 to enable the control system 20 to locate said surgical training tool 36 when the latter is stored.

This is why the present invention operates, in a kit, with a series of surgical training tools 36 (cf. FIG. 2). Hence, the surgical training kit thus formed (cf. FIGS. 5 and 6), comprises a modular surgical training platform 16 according to the present invention and at least one surgical training tool 36 configured to be connected to the haptic controller 18 of said platform 10. The kit according to the present invention may include two types of surgical training tools: the so-called “simple” tools and the so-called “complex” tools. The complex tools are complex electronised tools which embed a microcontroller capable of communicating directly with the control system 20.

These simple or complex surgical training tools 36 are modified surgical tools or copies of these. Thus, the simulation enabled by the platform 10 according to the present invention makes all or part of the physical actions to which these objects are subjected match with behaviours of the virtual twins in the virtual environment 100 displayed by the display unit 12 (cf. FIGS. 5 and 6).

As shown in the embodiment illustrated in FIGS. 2 and 3, the haptic controller 18 of each training module 16 comprises a swivel arm robot, the swivel arm robot having a free end intended to cooperate with the connection system 35.

The connection system 35 of the haptic controller 18 is universal, meaning that it allows connecting at least two different surgical training tools 36 (cf. FIG. 2).

More specifically, the connection system 35 comprises at least one key-lock type locking connector 35a configured to be fastened to the haptic controller 18 and to any surgical training tool 36 so as to ensure the removable connection thereof (cf. FIGS. 3, 9A and 9B). The connectors 35a, 35b also enables a transmission of the rotation according to the axis of the end of the haptic controller 18 towards the training module 16. As shown in FIGS. 3, 4A and 4B, the connection system 35 according to this embodiment comprises two key-lock type locking connectors 35a, 35b: a first connector 35a fastened to the haptic controller 18 and configured to cooperate in a removable manner with a corresponding second connector 35b fastened to the surgical training tool 36. Both connectors 35a, 35b can be reversibly fastened, either to the haptic controller 18, or to the surgical training tool 36. The two connectors 35a, 35b are obtained by 3D printing, either by layer deposition, or by sintering. In the example of FIGS. 9A and 9B, FIG. 9A shows the first connector 35a (herein the lock) of the haptic controller 18 and FIG. 9B shows the second connector 35b (herein the key) intended to cooperate with the surgical training tool 36. Fastening of the first connector 35a on the haptic controller 18 may be done in several ways. In the case of the illustration 9A, a jack interface already present on the haptic controller 18 originally has been used to fasten it. In other embodiments, it is possible to consider gluing the first connector 35a where it is adapted to another haptic controller 18 by creating an interface form specific to the latter. Preferably, fastening of the second connector 35b to the surgical training tool 36 is done by gluing. This primarily consists in gluing at the level of the distal rod of the surgical training tool 36. The 3D model of the second connector 35b is adapted by providing, on the face that is not visible in FIG. 9B, for a hole corresponding to the end of the distal rod 360 of the surgical training tool 36. The distal rod is then glued in the second connector 35b, for example by means of epoxy. This manufacturing process is not the only one that could be implemented. According to alternative embodiments, it is also possible to imagine 3D printing a reproduction of a surgical training tool 36 whose model would contain the second connector 35b.

The key-lock type connection 35 ensures a collinearity constraint according to the X axis of the connector 35a of the free end of the haptic controller 18. Hence, the two connectors 35a, 35b (and therefore the surgical training tool 36 and the haptic controller 18) are fully constrained according to all directions.

In order to stabilise the reversible connection between the haptic controller 18 and the surgical training tool 36, the connection system 35 may include, on each side of the “key-lock” system, at least one magnet 38 (cf. FIG. 9A, 9B). Preferably, the used magnets 38 consist of cubic magnets having a magnetisation force of 1.1 kg. This value allows for both a solidity of the attachment but also an easy disconnection of the surgical training tool 36, preferably in order to create a “plug and play” interface. Indeed, the magnets 38 ensure a contact constraint between the two connectors 35a, 35b. As mentioned hereinabove, the two connectors 35a, 35b are fully constrained in all directions, except in the direction collinear with the axis of the end of the haptic controller 18. Hence, the magnetisation allows constraining/holding the connection also in this direction. Nonetheless, the breaking force of this stress (and therefore breaking of the “key-lock” connection) is lower according to the axis X because the force of the magnets 38 are not very high. Hence, the obtained result is that the two connectors 35a, 35b are separated by pulling on the surgical training tool 36 more strongly than what is required to train the movement of the haptic controller 18. Thus, the haptic controller 18 should therefore be held in order to disconnect the surgical training tool 36. The presence of magnets 38 facilitates the connection and the disconnection between the surgical training tool and the haptic controller 18. Indeed, the magnets 38 enables a simple connection/disconnection gesture (of the “plug and play” type) without any screw or slide: the user approaches the surgical training tool 36 of the haptic controller 18 and the latter is connected alone by the action of the magnets 38.

Thus, the key-lock type connection system 35 according to the present invention has three distinct and complementary functions:
it enables an easy and “plug and play” attachment of the surgical training tool 36 using magnets 38,
it allows transmitting the rotational movement according to a central axis to the haptic controller 18,
in some cases, it allows electrically connecting a surgical training tool 36 to the training module 16.

Thus, the connection system 35 has an electronic component. Thus, each of the connectors 35a, 35b includes an aperture, a groove or a recess 37 intended for the insertion of an electrical connector (not shown in FIGS. 9A, 9B). In a preferred embodiment, this electrical connector is an electrical connector JST but other types of electrical connectors may be used. Preferably, the male portion of the electrical connector is inserted into the aperture 37 of the first connector 35a fastened to the end of the haptic controller arm 18. In this embodiment, the female portion of the electrical connector is inserted into the aperture 37 of the second connector 35b of the surgical training tool 36. Afterwards, as shown in FIGS. 2 and 3, a cable 39 connects the pins of the electrical connector to the control system 20. Another advantage of the connection system 35 according to the present invention is the simplicity with which it is possible to change the surgical training tool 36 to the haptic controller 18. This change should be simple and quick in order not to clutter up the learning with complex handling operation. Hence, it is necessary to provide a “plug and play” device, like the present invention.

Each haptic controller 18 is further configured to measure each movement in space of the surgical training tool 36 once the latter is connected to the haptic controller 18. Thus, each haptic controller 18 is provided with at least one external translation or rotation sensor 19 fastened on the different movable elements of the haptic controller 18 (cf. FIG. 1), so as to capture the three-dimensional position and orientation of any object connected to the haptic controller 18 of the training module 16.

Two categories of motion could be distinguished:
those that could be referred to as external, common to all surgical training tools 36, and which correspond to the three-dimensional position and orientation of the surgical training tool in space, and
those that could be referred to as internal, specific to some surgical training tools 36 so-called complexes, having a standby state and at least one activation state, such as the pull of a trigger or the rotation of an element and including an on-board electronic board.

These categories correspond to the two types of surgical training tools included in the surgical training kit according to the present invention.

The haptic controller 18 according to the present invention allows measuring the external movements (movements in space) of each connected surgical training tool 36.

In the case of complex surgical training tools 36, the connection system 35 may also have one or more other role(s) than the identification of the surgical training tool 36 connected to the haptic controller 18:
it could enable the recovery of the information on the movement of elements specific to the tool, such as the action of a trigger, for example. Moreover, and/or
it could enable the power supply of the internal electronics of the surgical training tool 36 connected to the haptic controller 18,
it could enable an electronic communication between the surgical training tool 36 and the training module 16.

Since the modularity of the platform 10 according to the invention allows connecting several training modules 16 together, to the calibration module 14 and to the control system 20, the platform 10 thus allows determining the positioning in space of several surgical training tools 36 connected to the different haptic controllers 18. If the control console 26 includes several training modules 16, the platform 10 enables the determination of the positioning of several surgical training tools 36 simultaneously, as soon as these are connected to a haptic controller 18.

The control system 20 of the platform 10 according to the invention further includes a system 40 for recognising each surgical training tool 36. The recognition system 40 of the surgical training tool 36 being configured to:
read the voltage derived from a voltage divider bridge specific to each surgical training tool 36,
communicate with the control system 20, so that the control system 20 recognises each surgical training tool 36 connected to the haptic controller 18.

Indeed, it is necessary to identify the surgical training tool 36 which is connected to the haptic controller 18 to enable the control system 20 to generate a corresponding movable virtual surgical element 102 in the virtual space 100.

Depending on considered the surgical training tool 36, the platform 10 uses a wireless connection and/or an electrical connection to identify the connected surgical tool (cf. FIG. 3).

In the case of a simple surgical training tool 36, the recognition system 40 comprises a microcontroller 42 preferably located in the base 30 of the training module 16, as shown in FIG. 7. The microcontroller 42 is connected to the surgical training tool 36 by means of the connection system 35, by the cable 39. In the case of a simple surgical training tool, the recognition system 40 further comprises, at the connection system 35 between the tool 36 and the haptic controller 18, an electronic device such as a voltage divider bridge in order to enable the recognition of the surgical training tool 36.

In the case of a complex surgical training tool, the recognition system 40 of the control system 20 recovers and analyses the information originating from the microcontroller of the complex surgical training tool 36. In this case, the wireless communication is enough for the identification.

The control system 20 of the platform 10 is configured to:
identify the different modules 14, 16, 21, 23, 25, 28 connected together either through a so-called electronic channel, or through a so-called software channel (as detailed hereinabove),
receive and analyse the data related to the movement(s) of each surgical training tool 36 connected to a haptic controller 18,
generate the virtual environment 100,
interface each movable virtual surgical element 102 of the virtual environment 100 with a corresponding real element.

Thus, the control system 20 generates a virtual image of each surgical training tool 36 connected to a haptic controller 18.

As already mentioned hereinabove, the virtual environment 100 also includes decorative elements 106 which cannot be moved and/or cannot be handled. For example, it may consist of an endoscopy screen 108 or a lamp which can be virtually handled by the users with, for example, a click on a button to turn them on. These decorative elements 106 do not have corresponding real elements.

Based on the information received from the recognition system 40 and the information collected at the haptic controller 18, the control system 20 is configured to transform/reproduce each movement in space of each surgical training tool 36 connected to a haptic controller 18 of the control console 26 into a corresponding virtual movement of its virtual image 102 in the virtual environment 100.

In the particular case of a connection with a simple surgical training tool 36, the control system includes:
a measuring unit (or microcontroller 42) configured to:
   identify the connected tools 36 and/or modules 14, 16, 21, 23, 25,
   gather the data of eigenmovements (or internal movements) of the connected surgical training tool 36,
a central unit configured to:
   generate the virtual environment 100,
   receive and analyse the data related to the movement(s) of each connected surgical training tool,
   interface each virtual surgical tool 102 of the virtual environment 100 with a corresponding real element, and possibly
   identify the connected modules 14, 16, 21, 23, 25.

In this particular case, as illustrated in FIGS. 1 and 7, the measuring unit (or microcontroller 42) is part of the training module.

The control system 20 is further configured to generate a feedback signal enabling the haptic controller 18 to generate in turn a corresponding haptic signal, according to what happens in the virtual environment 100.

In the present application, the “haptic signal” concept should be understood as a signal actively generated by the platform 10 according to the present invention. It should be differentiated from the “tactile feedback” concept, which is a mere passive feedback, automatically generated by the human body in response to handling of animate or inanimate objects.

Thus, the control system 20 causes the haptic controller 18 to generate a specific haptic feedback when the virtual tool 102 corresponding to the surgical training tool 36 handled by the user comes into contact with another virtual tool 102 or another virtual element such as a decorative element 106, of the virtual environment 100. This allows accentuating the immersive aspect of the simulation and providing an enhanced sense of reality; the interactions visible in the virtual environment 100 are also felt by the user.

In some embodiments, some technical elements of some complementary tooling modules 23 or of some anatomical modules 21 may also be configured to produce a haptic signal in response to some stimuli or situations. In other words, some anatomical modules 21 and some complementary tooling modules 23 are configured to generate a haptic signal in response to a stimulation of the control system 20 or an action of the user.

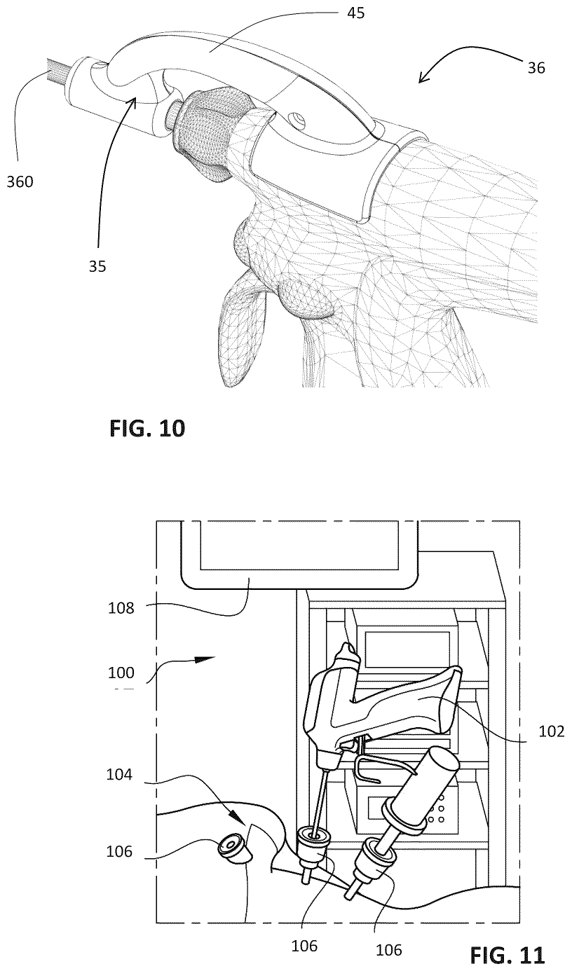

As already mentioned hereinabove, the present invention also comprises a kit formed by the platform 10 according to the invention and a surgical training tool 36. Some of these complex surgical training tools 36, like, for example, that one shown in FIG. 10, include a rotary distal rod 360. Thus, these tools 36 have a knob enabling rotation of the distal rod 360 and therefore of their axis. When this distal rod 360 is connected to the connection system 35, it is then impossible, for the control system 20 to measure/determine both the position in space of the surgical training tool 36 and the specific rotation of the distal rod 360: Indeed, the general rotation of the surgical training tool 36 relative to the axis of the haptic controller 18 should be transmitted so that its virtual twin (virtual tool 102) could be oriented similarly in the virtual environment 100 without losing the specific rotation of the distal rod 360 induced by the operation of the surgical training tool 36.

To solve this problem, the connection system 35 has a particular embodiment with an arcuate part 45. As shown in FIG. 10, the arcuate part 45 enables the free rotation of a rod orienting wheel on the surgical training tool 36 yet without losing the information on the orientation of the tool 36 itself. The arcuate part 45 is fastened on the grippable body of the tool 36 on the one hand and on the distal rod 360 secured to the second connector 35b on the other hand. The distal rod 360 is cut so that the portion under the arcuate part 45 could be freely rotated without any consequence on the rotation at the level of the key-lock mechanism of the connection system 35, at the end of the haptic controller 18.

Preferably, the arcuate part 45 is printed using a layer deposition 3D printer, but any other plastic manufacturing process may be used, for example laser sintering. Preferably, the arcuate part 45 is designed in two portions in order to be able to be easily dismounted, the two portions are assembled by means of screws.

Thus, one could notice that the platform 10 according to the invention is built around the training module(s) 16. Thus, each training module 16 is a central element located at the convergence of the different elements of the platform 10 according to the present invention. As already mentioned, each training module 16 is organised around a base 30 which allows fastening the different elements:
the microcontroller 42 and its connection cable 39 to a connection system 35 intended to connect the surgical training tool 36 to the control system 20,
a haptic controller 18 including a robot for acquiring a three-dimensional movement by polar referencing,
magnets 34, 38, and, possibly
one or more electrical connector(s) (for example connectors with retractable pins as seen hereinabove).

Once connected to the virtual reality display unit 12, by means of the calibration module 14, these different joined elements allow relating, in a surgical procedure simulation context, the handling of physical surgery objects to their virtual twin in a virtual reality simulation. The platform 10 enables a positioning of these objects in space and an identification of the movements to which the physical object is subjected, enabling a user to best get the feelings induced by a surgical procedure without subjecting him/her to unavoidable stress or hazard.

1-15. (canceled)

16. A modular surgical training platform configured to interface a virtual environment comprising at least one movable virtual surgical element, the training platform comprising:
a virtual reality display unit configured to display, to a user, the virtual environment,
a calibration module connected to the virtual reality display unit,
at least one training module, each training module including a haptic controller,
a control system configured to:
identify the different modules connected together,
generate the virtual environment,
interface each movable virtual surgical element of the virtual environment with a corresponding real element,
wherein all of the modules are configured to be reversibly attached to each other in a known configuration,
wherein each haptic controller includes a connection system configured to reversibly mechanically connect a surgical training tool, each haptic controller being further, configured to measure each movement in space of the surgical training tool once the latter is connected to the haptic controller,
wherein the control system further includes a system for recognising each surgical training tool configured to obtain identification information specific to the connected surgical training tool, and communicating the identification information to the control system, so that the control system recognises each surgical training tool connected to the haptic controller,
wherein the control system generates a virtual image of each surgical training tool connected to the haptic controller,
wherein the control system is configured to receive and analyse the data related to the movement(s) of each surgical training tool connected to the haptic controller, and reproducing each movement of the surgical training tool connected to the haptic controller into a corresponding virtual movement of its virtual image in the virtual environment,
wherein each virtual movement is made visible to the user by the display unit.

17. The surgical training platform according to claim 16, wherein the control system includes:
a measurement unit configured to:
identify the connected surgical training tools and/or modules,
gather the movement data of the connected surgical training tool,
a central unit configured to:
generate the virtual environment,
receive and analyse the data related to the movement(s) of each connected surgical training tool,
interface each movable virtual surgical element of the virtual environment with a corresponding surgical training tool.

18. The surgical training platform according to claim 17, wherein the measuring unit is part of the training module.

19. The surgical training platform according to claim 16, wherein the control system is further configured to generate a feedback signal enabling the haptic controller to generate a haptic signal when at least two virtual elements interact in the virtual environment.

20. The surgical training platform according to claim 16, further comprising at least one anatomical module configured to represent or reproduce all or part of a human anatomical portion.

21. The surgical training platform according to claim 20, wherein the anatomical module is configured to generate a haptic signal in response to a stimulation of the control system or an action of the user.

22. The surgical training platform according to claim 16, further comprising at least one complementary tooling module configured to represent or reproduce all or part of a tooling likely to be present in an operating room or all or part of a surgical tool required during a surgical procedure but not intervening directly on the body of a patient.

23. The surgical training platform according to claim 22, wherein the complementary tooling module is configured to generate a haptic signal in response to a stimulation of the control system or an action of the user.

24. The surgical training platform according to claim 16, further comprising a storage module configured to enable storage of one or more surgical training tool(s).

25. The surgical training platform according to claim 16, further comprising at least one spacer module configured to connect two modules (in an indirect manner.

26. The surgical training platform according to claim 16, wherein the at least one spacer module is configured to arrange two modules on distinct horizontal planes.

27. The surgical training platform according to claim 16, wherein each module comprises a base having a specific shape, the shapes of the different bases of the different modules being complementary to each other, so as to obtain a stable and adapted interlocking of the different modules.

28. The surgical training platform according to claim 16, wherein the connection system of the haptic controller allows connecting at least two different surgical training tools.

29. The surgical training platform according to claim 16, wherein the haptic controller of the training module comprises a swing arm robot, the swivel arm robot having a free end intended to cooperate with the connection system.

30. A surgical training kit comprising the modular surgical training platform according to claim 16 and at least one surgical training tool configured to be connected to the haptic controller of the platform.
